(19) United States
(12) Patent Application Publication
(10) Pub. No.: US 2011/0189242 A1
(43) Pub. Date: Aug. 4, 2011

(54) ORAL GALENIC FORM, POLYMER PRODUCTION METHOD AND USE OF SAME

(75) Inventors: Thierry Vandamme, Strasbourg (FR); Mustafa Beesh, Strasbourg (FR); Paulina Majewska, Wroclaw Pologne (PL)

(73) Assignees: UNIVERSITE DE STRASBOURG, Strasbourg (FR); CENTRE NATIONAL DE LA RECHERCHE SCIENTIFIQUE, CNRS, Paris (FR)

(21) Appl. No.: 12/997,743

(22) PCT Filed: Jun. 11, 2009
(86) PCT No.: PCT/FR09/00706

§ 371 Date: Mar. 10, 2011

(30) Foreign Application Priority Data
Jun. 12, 2008 (FR) 08/03292

(51) Int. Cl. A61K 9/00; A61K 47/30

(52) U.S. Cl. 424/400; 514/772.3

(57) ABSTRACT

The polymers, according to the invention, comprise a polysaccharide block (17) and several hydrophobic polyacrylic blocks (18) enabling the polymer to remain intact until the polymer reaches the colon. The polysaccharide block is degraded by the colonic microflora irrespective of the pH, while the polyacrylic blocks are solublilized at neutral pH. Colon-specific release of the active ingredients is thus provided in all cases. These polymers may serve as a coating in particular for tablets, gel capsules, granules or microgranules, or serve as matrix agents in the preparation of such pharmaceutical forms. This invention concerns the fields of medicine, pharmacy and dietetics.

This application is a national stage completion of PCT/FR2009/000706 filed Jun. 11, 2009 which claims priority from French application Ser. No. 08/03292 filed. Jun. 12, 2008.

### FIELD OF THE INVENTION

The present invention concerns a new oral galenic form, preferably pharmaceutical, for targeting the colon with doses of active principals. More particularly, the invention relates to new polymers for targeting the colon with active principals irrespective of its pH because these polymers are degraded specifically by colonic flora. These new polymers may be used to form a coating or to serve as matrix agents.

More particularly, the invention relates to new polymers for targeting the colon with active principals irrespective of its pH because these polymers are degraded specifically by colonic flora. These new polymers may be used to form a coating or to serve as matrix agents.

These new polymers may be used to form a coating or to serve as matrix agents.

### BACKGROUND OF THE INVENTION

In current practice, oral administration is undeniably the preferred and easiest way to dispense medication.

With certain medications it is of interest, or even mandatory, for the active principals they contain to be released within the colon, which constitutes the last portion of the digestive tract.

This is the case, for example, with active principals designated for local treatment of colon pathologies such as, for example, inflamed colon, ulcerated colon, colon cancer, Crohn's disease, and others.

More generally, the colon is the preferred absorption site for peptides, proteins, and numerous other active principals targeting various pathologies, specifically asthma, angina, arthritis, located in other areas of the body.

To improve the effectiveness of these medications and prevent their active principals from being released too quickly or degrading in the stomach before reaching the colon level, a solution is sought to protect these active principals during their trajectory through the first portion of the digestive tract and then selectively release them in the colon.

This would allow the dose of the active principal to be decreased, limiting it to only the amount necessary in the colon, thereby reducing the cost of the medication as well as its secondary effects.

To reach this goal, the prior art has explored several paths.

The first consists of providing an external coating protecting the active principal, resistant to the acidic milieu of the stomach and to intestinal enzymes, and arriving intact in the colon. This coating must then degrade within the colon to release the active principals contained in the medication.

Since the colon normally has an essentially neutral pH, coatings now in use are designed to resist the acid pH in the stomach and to dissolve at a neutral pH so as to release the active principal of the medication only within the colon.

However, with a great many illnesses present at this level of the digestive tract and with chronic colon inflammation such as that encountered in Crohn's disease, for example, colon pH decreases and becomes acidic.

Conventional pH-sensitive coatings that are formulated to resist acidic conditions in the stomach therefore do not dissolve in the colon when it has become acidic and the active principals of medications with these coatings are not released.

In such situations, which involve clinical cases where targeting the colon with the dosage is the most desirable, conventional pH-sensitive coatings are not effective and do not allow the active principals to be selectively released within the colon.

In order for them to be independent of pH conditions within the colon, the prior art has used coatings that degrade slowly to delay the release of active principals over time. This type of coating is not satisfactory, however, for release within the colon because the coating's degradation time is imprecise when it needs to be very long, since release occurs within the colon, located in the last portion of the digestive tract.

Another approach consists of using multilayer coatings such that each polymer is degraded in succession by the different milieus it encounters after the medication is ingested and such that the last layer degrades within the colon.

Complex, tedious, and unreliable, this solution has not been put into practice, notably because it is difficult to reproduce the application of several polymers onto one pharmaceutical form and because there is no guaranty that the last polymer coating will degrade irrespective of the colon's pH.

In addition, a solution has been proposed based on the technique of azoic hydrogels that consists of incorporating the active principals into acrylic polymers reticulated by azoic connections, the azoic connections being degraded in a specific way by colonic bacteria to form hydrosoluble linear polymers.

However, this solution does not seem to be of interest due to the probable toxicity of the polymers used and/or resulting from the degradation (presence of amine functions) and because of the great difficulty of incorporating active principals within the reticulated polymer at the outset.

One final path studied by the prior art consists not of a galenic transformation, but rather a chemical modification of the one or more active principals so as to obtain compounds called prodrugs, incapable of absorption at the level of the stomach and the small intestine. These chemical modifications principally comprise the formation of azoic compounds or grafting sugars onto steroids. During their passage through the colon, these prodrug compounds are reduced by the colonic enzymes and bacteria and thus discharge the pharmacologically active principals.

However, formation of these prodrugs is only possible if the initial active principal contains a thiol or amine function. With the vast majority of active principals, which do not possess these chemical functions, it is impossible to use such a method.

Therefore, a great need exists for a coating without the drawbacks described above and which would allow the colon to be targeted with active principals irrespective of its Ph.

### SUMMARY OF THE INVENTION

The objective of the invention is to furnish new polymers for targeting the colon with doses of active principals. These polymers degrade specifically within the colon and remain intact until they arrive at this point along the digestive tract. For the same reason, they do not degrade in the mouth, the stomach, or the small intestine.

In addition, these polymers are not toxic and the byproducts of their degradation are not harmful to health.

To overcome this technical problem, the present invention teaches a new polymer for colon-specific release which protects at least one active principal contained in an oral galenic form until it reaches the colon, specifically a pharmaceutical, and releases it specifically within the colon. This polymer remains intact in the first portion of the digestive tract, that is, the mouth, esophagus, stomach and small intestine, and degrades at the level of the colon.

This polysaccharide based polymer undergoes enzymatic degradation upon contact with colonic microflora. This degradation is independent of pH and is always possible because colonic bacteria and enzymes are always present in sufficient quantity within the colon, even if its pH has changed.

However, since polysaccharides are hydrosoluble, they dissolve prematurely in the mouth and the stomach. Surprisingly, and despite being based on hydrosoluble polysaccharides, the polymers according to the invention are sufficiently hydrophobic that they do not degrade in the mouth and the beginning of the digestive tract. For this reason, the invention relies on chemical modification of polysaccharides in order to render them more hydrophobic.

Therefore, the novel polymer according to the invention is a block copolymer comprising a polysaccharide block designed to be degraded by colonic enzymes and bacteria irrespective of the pH within the colon, and at least two hydrophobic polyacrylic blocks grafted one after the other onto the polysaccharide block, allowing the polymer to remain intact until it reaches the colon level.

Advantageously, the polyacrylic blocks of the polymer according to the invention solubilize when the pH is approximately 7, permitting faster dispersion of the active principals when the colon's pH is normal.

Thus, the polymers of the invention advantageously release active principals at colon level using two different mechanisms: first, degradation of the polysaccharide block sensitive to colonic bacteria, which are produced in every instance; and second, by solubilizing polyacrylic blocks at the neutral pH resulting from normal pH condition within the colon. The polymers of the invention, therefore, can be used “universally,” whatever the patient's condition.

Preferably, the polymers of the invention comprise two or three polyacrylic blocks, each of these blocks preferably being a chain of methyl polylacrylate, methyl polymethacrylate, or polymethacrylic acid.

Advantageously, the block polymers of the invention may be used to create a coating for oral galenic forms, preferably pharmaceutical. They may also serve as matrix agents for the preparation of such oral galenic forms.

Thus, the invention furnishes oral galenic forms containing at least one active principal that must be delivered specifically to the colon and containing at least one polymer according to the invention for colon-specific release. Examples of these oral galenic forms are tablets, gel capsules, granules, microgranules, and the like.

The invention also teaches a method for producing such a polymer for colon-specific release wherein the polyacrylic blocks are obtained by successively adding different acrylic monomers to the polysaccharide block during several successive steps of radical polymerization in emulsion.

### BRIEF DESCRIPTION OF THE DRAWINGS

Other characteristics and features of the invention will be apparent from reading the following detailed description, taken in conjunction with the attached drawings, in which:

FIG. 1 is a schematic view of the trajectory of a medication according to the invention through a patient's digestive tract with the active principals being released at colon level;

FIG. 2 is a schematic cross-sectional view of a tablet having a polymer-based coating according to the invention;

FIG. 3 is a schematic cross-sectional view of a granule having a polymer-based coating according to the invention;

FIG. 4 is schematic cross-sectional view of a gel capsule containing granules each having a polymer-based coating according to the invention;

FIG. 5 is a schematic cross-sectional view of a gel capsule with its exterior wall having a polymer-based coating according to the invention;

FIG. 6 is a schematic cross-sectional view of a tablet having a polymer according to the invention as the matrix agent;

FIG. 7 is a schematic cross-sectional view of a gel capsule containing granules comprising a polymer according to the invention as the matrix agent;

FIG. 8 corresponds to the general chemical formula for one example of a polymer according to the invention;

FIG. 9 is a schematic block representation of a polymer according to the invention;

FIG. 10 is a schematic representation of the reaction for synthesizing a polymer according to the invention; and

FIGS. 11-16 are graphs illustrating the release over time of an active principal contained in three series of tablets having a polymer-based coating according to the invention, placed in an experimental environment simulating in vitro the trajectory through the human digestive tract.

### DETAILED DESCRIPTION OF THE DRAWINGS

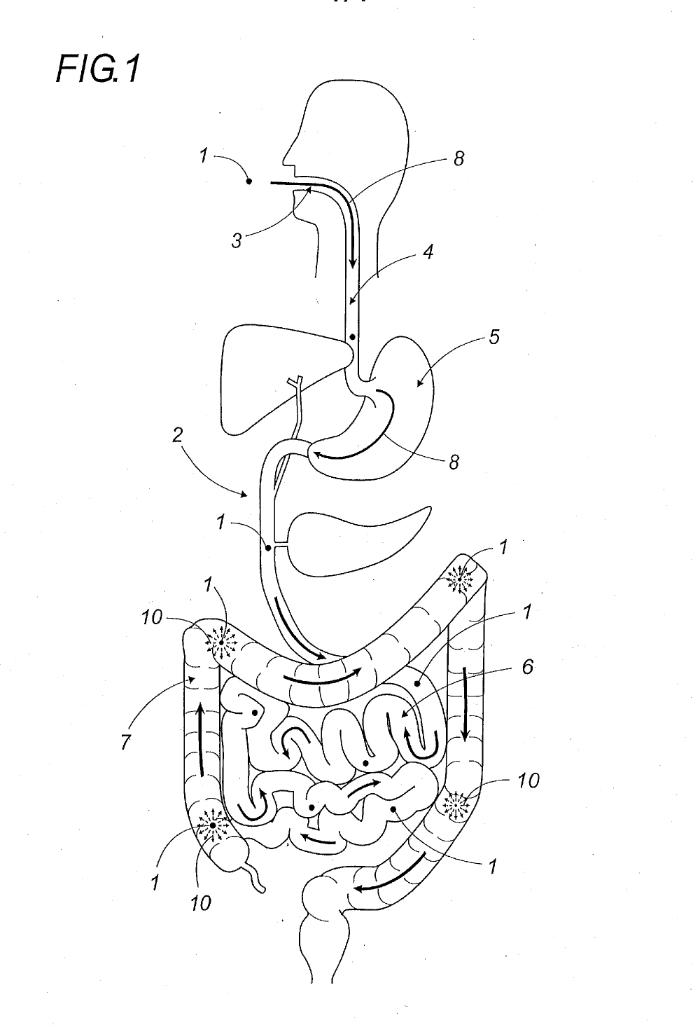

The novel polymer for an oral galenic form according to the present invention will now be described in detail with reference to FIGS. 1 through 16.

The polymer of the invention may be used to protect any active principal of a medication, whether it is in the form of tablets, gel capsules, granules, microgranules, or any other oral galenic form.

The preferred domains for application of the polymer according to the invention are medicine and pharmaceuticals. However, its application is not limited to these domains and may extend notably to phytotherapy, homeopathy, dietetics, cosmetology, nutrition, or the veterinary or animal husbandry fields.

The polymer of the invention may be used whenever it is necessary, desirable, or of interest to aim the release of active principals at the colon, regardless of the nature of the active principal or its function.

For example, it may be used in manufacturing medication, diagnostic products, nutritional complements, vitamin-based products, minerals, oligo elements, or any other appropriate product conceived by a person skilled in the art.

FIG. 1 is a schematic representation of the path taken by an oral galenic form or medication 1 according to the invention along the digestive tract 2 of a patient.

After being ingested by the patient, the medication 1 passes successively through the mouth 3, the esophagus 4, the stomach 5, the small intestine 6, and finally the patient's colon 7. Its trajectory through the digestive tract is represented by large black arrows 8.

Advantageously, medication 1 according to the invention remains intact throughout the first portion of the digestive tract until it arrives at the level of the colon 7, where it begins to release the one or more active principal (5) which it contains.

In this drawing the diffusion of active principal 9 has been symbolized by small dotted line arrows 10. Medication 1 remains in the colon for a long period of time, specifically ranging from 24 to 72 hours, advantageously ensuring that all of the active principal is released within the colon.

Depending on the situation, medication 1 of the invention may be designed so that the active principal is released either quickly and completely upon entering the colon, or so that it is released slowly and progressively throughout its path through the colon.

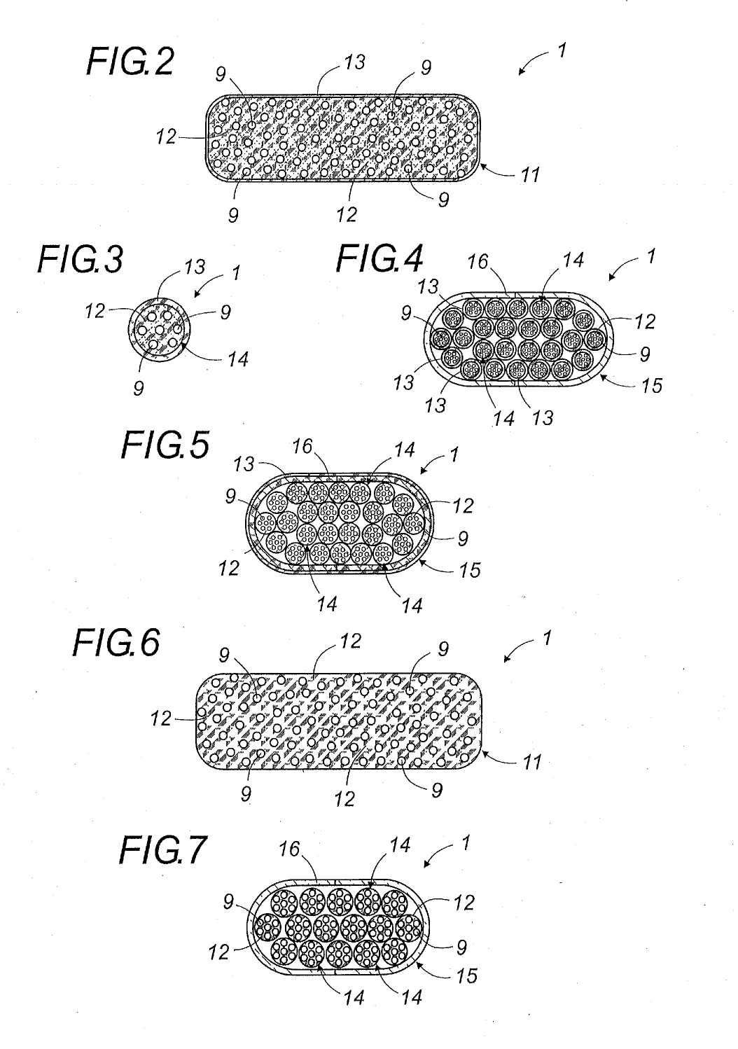

Medication 1 according to the invention may assume various galenic forms for oral administration. Several examples of suitable oral forms are shown in FIGS. 2 through 7. Obviously, a person skilled in the art might conceive of many others without exceeding the scope of the present invention.

In these drawings active principal 9 is depicted schematically by small white circles.

In FIG. 2 the medication 1 is shown in the form of a tablet II. This tablet 11 is manufactured in the conventional way from a mixture of active principal 9 and one or more excipients 12, compressed to form tablet 11.

The one or more excipients 12 are compounds, generally solid and preferably pulverized, adapted for the formation of tablets. They may consist of any pharmacologically acceptable excipient with properties suitable for this purpose, notably one or more matrix agents possibly mixed with other excipients of a different nature. There are very many examples of excipients in the prior art, notably lactose, microcrystalline cellulose, cellulosic derivatives, gums, and the like.

In order to protect active principal 9 until it arrives in the colon, tablet 11 is covered with a coating 13 formed of a layer of polymer according to the invention completely covering the exterior surface of tablet 11. The thickness of this coating 13 preferably ranges from 1 μm to 1 mm, with a value preferably ranging from 100 μm to 200 μm.

Advantageously, the speed at which active principal 9 is released in the colon can be controlled by varying the thickness of coating 13. However, coating 13 must be sufficiently thick to protect the active principal until it reaches the colon, while preventing any prior diffusion of active principal through coating 13.

Medication 1 may also take the form of one or more granules 14 or microgranules, as shown in FIG. 3.

Like tablet 11, granule 14 is composed of one or more excipients 12 used to support active principal 9. This time excipient 12 is adapted for the formation of granules 14 and the technique used for this formation. The same compound or the same mixture of components may be used as for manufacturing tablets 11, or one or more components specific to this application may be used.

Granule 14 may be obtained using one or more methods widely known by persons skilled in the art, some examples of which are provided below. Excipient 12 such as a matrix agent and active principal 9 may simply be mixed, for example, before being shaped by compression. Active principal 9 may also be absorbed and/or adsorbed on crystals or grains of excipient 12. According to yet another technique, a paste-like mixture of excipient 12 and active principal 9 may be shaped by extrusion, cutting, and forming the resulting pieces into spheres.

The entire exterior surface of granule 14 is then covered with polymer coating 13 according to the invention, protecting active principal 9 until it reaches the colon.

If they are small or if it is necessary to facilitate administration to the patient, these granules 14 or microgranules maybe grouped inside gel capsules 15 preferably in an amount corresponding to one unit dose of the medication 1.

As shown in FIGS. 4 and 5, gel capsule 15 consists of an envelope 16, preferably gelatin-based, formed of two interlocking pieces that enclose a certain number of granules 14.

According to the variation in FIG. 4, these granules 14 may be covered, each individually, with a protective polymer-based coating 13 according to the invention. Granules 14 are then grouped inside capsules 15 after having been separately coated. According to another variation shown in FIG. 5, polymer-based protective coating 13 according to the invention may cover the exterior surface of envelope 16 constituting gel capsule 15 rather than coating each granule 14 individually. Granules 14 are then simpler and contain only active principal 9 and one or more excipients 12. In this case the coating occurs subsequent to placing the granules in the gel capsule.

To protect the active principals contained in tablet 11, granule 14, microgranule, gel capsule 15, or any other appropriate oral form, until they reach the colon, coating 13 contains one or more polymers according to the invention. It may also contain other components adapted to form such a coating 13, specifically a plastifying element, an element whose structure is insoluble regardless of the pH it encounters, for example, a commercial polymer known as “Eudragit RS” or “Eudragit RL” used to stabilize the mechanical properties of the unit, or supplemental substances, talc or kaolin, for example, in order to lower the global selling price of coating 13 and to protect the photosensitive active principals by virtue of its white color, or any other conceivable substance adapted to form coating 13.

Another embodiment shown in FIGS. 6 through 7 consists of using a polymer according to the invention not as a coating 13, but as a matrix agent.

A polymer according to the invention can thus serve as an excipient 12 and more precisely as a matrix agent for manufacturing tablets 11 (FIG. 6), granules 14 (FIG. 7), microgranules isolated or grouped in gel capsules 15, or any other adapted form of oral galenic.

For this reason, the polymer according to the invention may be used alone or mixed with other matrix agents or excipients used in the pharmaceutical domain. A mixture of several polymers according to the invention may also be used.

Since active principal 9 is surrounded with the polymer of the invention at the core of the galenic form, a peripheral coating is no longer necessary to ensure protection. Nevertheless, such a coating may be provided, whether it is polymer-based according to the invention or some other constituent.

In order to protect active principal 9, the invention teaches a new polymer that is a block copolymer, that is, a polymer having several different polymer chains connected to one another by covalent bonds.

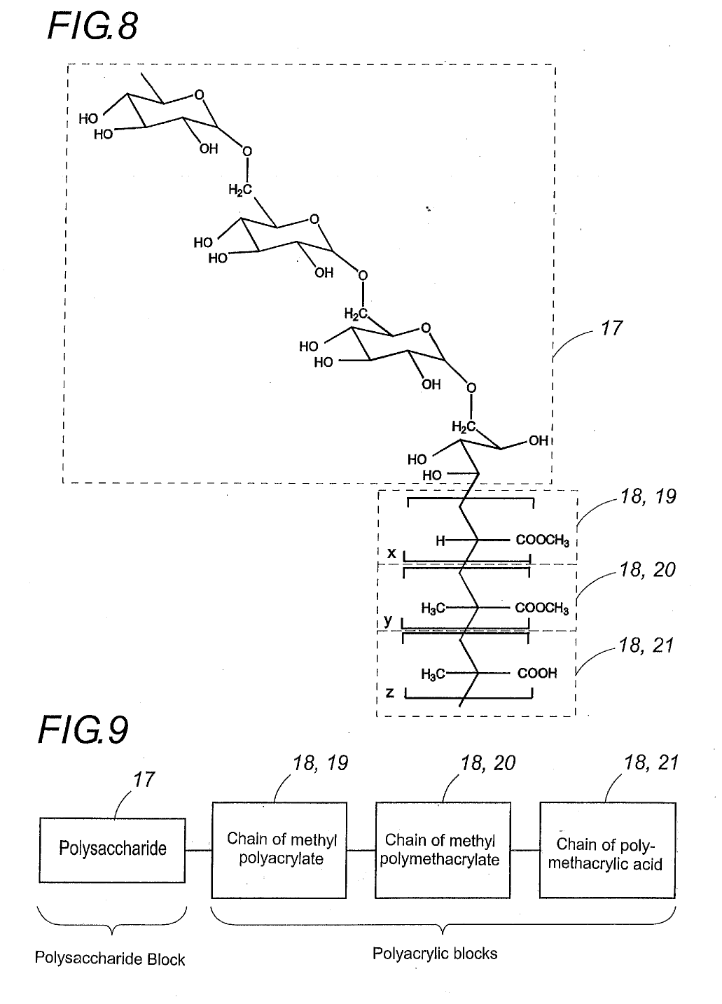

The chemical formula for one example of a polymer according to the invention is shown in FIG. 8. It is also represented schematically in FIG. 9 to better clarify the concept of blocks for the reader.

According to the invention, this copolymer consists of a polysaccharide block 17 and several synthetic polyacrylic blocks 18.

Polysaccharide block 17, which is hydrosoluble when isolated, is specifically degraded by colonic bacteria and enzymes regardless of Ph.

The block 17 is made from a polysaccharide macromolecule, preferably dextran, guar gum, amylose, amidine, chitosan, pectin, or any other molecule comprising sugars and having the characteristic of being easily and specifically degradable by colonic enzymes and bacteria. In the various attached drawings, polysaccharide block 17 is formed from dextran.

The polysaccharides are non-toxic macromolecules consisting of metabolites that occur naturally in the colon. Therefore, they do not pose any problems with toxicity, either when intact or decomposed.

The polymer according to the invention also comprises polyacrylic blocks 18 that are grafted to polysaccharide block 17 and whose hydrophobic properties advantageously prevent the polysaccharide block from dissolving in an aqueous milieu.

Thanks to these polyacrylic blocks 18, the polymer of the invention is sufficiently hydrophobic in its entirety that it does not dissolve too quickly in an aqueous milieu, preventing premature release of the active principal in the mouth, esophagus, stomach, or the first portions of the intestine.

Moreover, polyacrylic blocks 18 become solubilized at a neutral pH, thereby releasing the medication's active principal, protected by the polymer of the invention, more quickly when the pH levels in the colon are normal.

Acrylic polymers have been used for a long time in the pharmaceutical field and are known for their lack of toxicity. Thus, the polymer of the invention in its entirety, either when whole or after decomposition, is absolutely not toxic to the patient. Polyacrylic blocks 18 are preferably obtained by the successive radical polymerization of acrylic monomers onto polysaccharide block 17.

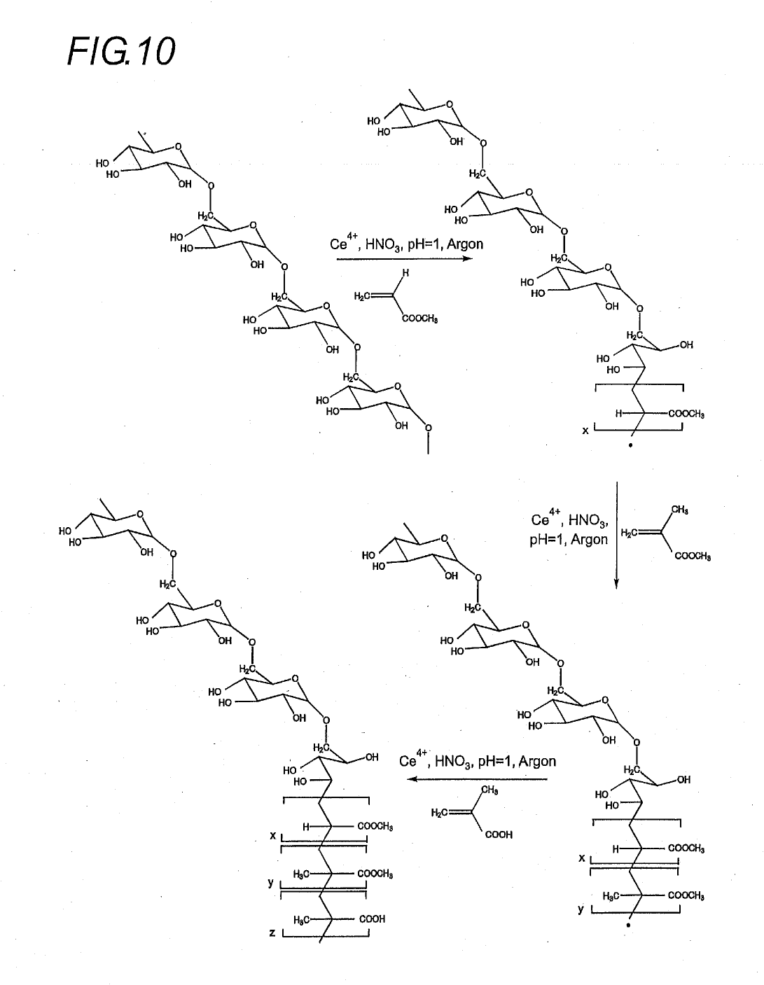

Therefore acrylic monomers can be successively added to the polysaccharide block using the radical method, as shown in FIG. 10. These acrylic monomers are preferably chosen from the following group: methyl acrylate, methyl methacrylate, and methacrylic acid.

To produce the polymer of the invention, these monomers must be added in succession and not as a mixture. A relative order among these monomers in relation to one another must also be respected. These monomers should therefore be added in the following relative order: methyl acrylate, methyl methacrylate, and methacrylic acid.

If these three monomers are added after polymerization, as shown, there is obtained from polysaccharide block 17 a first block 19 formed of a chain of methyl polyacrylate, a second block 20 formed of a chain of methyl polymethacrylate, and a third block 21 formed of a chain of polymethacrylic acid.

The last block 21 improves enzymatic and bacterial degradation of the polysaccharide block. In actuality polymethacrylic acid is ionized at a pH of 7 and contributes to hydrating the polymer more easily, giving it the consistency of a hydrogel, which facilitates degradation of the polysaccharide portion thereafter by the colonic enzymes and bacteria.

The inventors have discovered that in order to resolve the technical problem, the synthetic portion of the copolymer must be composed of several different polyacrylic chains, preferably two or three. Preferably, the synthetic portion comprises three polyacrylic chains formed of three successive polymerizations of different acrylic monomers.

This number may vary but if it is too high, stearic obstruction may be too elevated for the enzymes and bacteria in the colon to reach the polysaccharide block in order to degrade it. Conversely, if the number is too low, the resulting copolymer may have insufficient hydrophobic properties.

Polymers according to the invention with two polyacrylic blocks 18 have also been synthesized. In this case, to follow the order previously cited, they may comprise following their polysaccharide block 17 a methyl polyacrylate block 19 and a methyl polymethacrylate block 20, or a methyl polyacrylate block 19 and a polymethacrylic acid block 21, or even a methyl polymethacrylate block 20 and a polymethacrylic acid block 21.

To obtain these block copolymers according to the invention, the synthesis process retained preferably consists of opening a polysaccharide block sugar and adding to it in succession the polyacrylic chains, one after the other, using several successive radical polymerization steps in emulsion.

With reference to FIG. 10, we will now address a preferred example of polymer synthesis according to the invention.

The first step consists of reacting dextran with methyl acrylate in the presence of cerium (IV) and argon in a highly acidic milieu (pH=1) obtained by adding nitric acid. To do this, it is preferable to begin by thoroughly dissolving the polysaccharide in the nitric acid (preferably 0.2M) in an atmosphere of argon at about 60° C., then adding to it the cerium and the first monomer.

The concentration of cerium (IV) preferably ranges from 0.001 and 0.02 mol/l, with the value preferably being generally equal to 0.016 mol/l.

During this step, a polysaccharide block sugar is opened and a first polyacrylic chain of methyl polyacrylate 19 is affixed to it using radical polymerization of methyl acrylate monomers.

During the second step, in the same way and under the same conditions, a new polyacrylic chain of methyl methacrylate 20 is affixed, which will bond following the first one, by causing the intermediate compound obtained in the preceding step to react with the methyl methacrylate.

Finally, in the third step, still in the same way and under the same conditions, a new polyacrylic chain of polymethacrylic acid 21 is affixed, which will bond following the preceding one, by causing the intermediate compound resulting from the preceding step to react with the methacrylic acid.

The final product is then purified, primarily to eliminate the cerium and the monomers that did not react. To do this, the solution obtained is inserted into a dialysis membrane allowing the polymer to separate from the monomers that did not react. Similarly, the cerium in solution is eliminated during this purification.

After drying, the resulting polymer is in powder form.

In use this powder is solublized, for example, or placed in suspension in a conventional pharmaceutical solvent, for example, tetrahydrofuran, methanol, or ethanol. It may then be pulverized on medication 1, alone or especially in addition to a plastifying agent such as, for example, ethyl citrate, butyl phthalate or even another polymer to form a coating 13.

To assist in understanding the invention, some examples of synthesis of several polymers according to the invention formed of different polysaccharides and/or different polyacrylic chains will be detailed below. These polymers have been obtained through radical polymerization in emulsion using the previously described method.

### Example 1

### Synthesis of a Block Polymer: Dextran 70-Methyl Polyacrylate-Methyl Polymethacrylate-Polymethacrylic Acid

2.2 g dextran 70 (PM=70000 Da) were dissolved in 32 ml of nitric acid at 0.2M in a 250 ml triple-neck flask equipped with a condenser and an agitator, with argon barbotage, and the flask plunged into a 60° C. water bath.

After 10 minutes 32 ml of a 0.08 M solution of ceric ammonium nitrate (IV) in 0.2M of nitric acid and 9.1 ml of methyl acrylate were added under high agitation.

After 20 minutes 12.5 ml of a 0.08M solution of ceric ammonium nitrate (IV) in 0.2M of nitric acid and 3.5 ml of methyl methacrylate were added under high agitation.

After 20 minutes 5 ml of a 0.08 M solution of ceric ammonium nitrate (IV) in 0.2M of nitric acid and 1.4 ml of methacrylic acid were added under high agitation.

Twenty minutes after the last addition, the argon barbotage was stopped and the reaction allowed to continue for 50 minutes under gentle agitation.

After cooling to ambient temperature, the resulting product was purified by dialysis and then lyophilized in order to obtain the polymer of the invention.

### Example 2

### Synthesis of a Block Polymer: Dextran 11-Methyl Polyacrylate-Polymethacrylic Acid

2.2 g of dextran 11 (PM=11000 Da) were dissolved in 32 ml of nitric acid at 0.2M in a 250-ml. triple-neck flask equipped with a condenser and an agitator, with argon barbotage, and the flask plunged into a 60° C. water bath.

After 10 minutes 35 ml of a 0.08M solution of ceric ammonium nitrate (IV) in 0.2M of nitric acid and 10 ml. of methyl acrylate were added under high agitation.

After 20 minutes 15 ml. of ceric ammonium nitrate (IV) solution and 4 ml of methacrylic acid were added under high agitation.

Twenty minutes after the last addition, the argon barbotage was stopped and the reaction allowed to continue for 50 minutes under gentle agitation.

After cooling to ambient temperature, the resulting product was purified by dialysis, and then lyophilized in order to obtain the polymer of the invention.

### Example 3

### Synthesis of a Block Polymer: Amylose-Methyl Polyacrylate-Methyl Polymethacrylate-Polymethacrylic Acid

2.2 g of amylose (corn amidine) were dissolved in 32 ml of nitric acid at 0.2 M in a 250 ml triple neck flask equipped with a condenser and an agitator, with argon barbotage, and the flask plunged into a 60° C. water bath.

After 10 minutes, 16.66 ml of a 0.08M solution of ceric ammonium nitrate (IV) in 0.2M of nitric acid and 4.66 ml of methyl acrylate were added under high agitation.

After 20 minutes, 16.66 ml of a 0.08M solution of ceric ammonium nitrate (IV) in 0.2M of nitric acid and 4.66 ml of methyl methacrylate were added under high agitation.

After 20 minutes, 16.66 ml of a 0.08M solution of ceric ammonium nitrate (IV) in 0.2M of nitric acid and 4.66 ml of methacrylic acid were added under high agitation.

Twenty minutes after the last addition, the argon barbotage was stopped and the reaction allowed to continue for 50 minutes under gentle agitation.

After cooling to ambient temperature, the resulting product was purified by dialysis, and then lyophilized in order to obtain the polymer of the invention.

### Example 4

### Synthesis of a Block Polymer: Amylose-Methyl Polyacrylate-Polymethacrylic Acid

2.2 g of amylose (corn amidine) were dissolved in 32 ml of nitric acid at 0.2M in a 250-ml. triple neck flask equipped with a condenser at 60° C. under gentle agitation and with argon barbotage.

After 10 minutes, 45 ml of a 0.08M solution of ceric ammonium nitrate (IV) in 0.2M of nitric acid and 12.6 ml of methyl acrylate were added under high agitation.

After 20 minutes, 5 ml of a 0.08 M solution of ceric ammonium nitrate (IV) in 0.2M of nitric acid and 1.4 ml of methacrylic acid were added under high agitation.

Twenty minutes after the last addition, the argon barbotage was stopped and the reaction allowed to continue for 50 minutes under gentle agitation.

After cooling to ambient temperature, the resulting product was purified by dialysis, and then lyophilized in order to obtain the polymer of the invention.

### Example 5

### Synthesis of a Block Polymer: Chitosane 70-Methyl Polymethacrylate-Polymethacrylic Acid

2.2 g of chitosane 70 were dissolved in 32 ml of nitric acid at 0.02M in a 250-ml triple neck flask equipped with a condenser and an agitator, with argon barbotage, the flask being plunged into a 60° C. water bath.

After 10 minutes, 35 ml of a 0.08M solution of ceric ammonium nitrate (IV) in 0.2M of nitric acid and 10 ml of methyl methacrylate were added under high agitation.

After 20 minutes, 15 ml. of a 0.08M solution of ceric ammonium nitrate (IV) in 0.2M of nitric acid and 4 ml of methacrylic acid were added under high agitation.

Twenty minutes after the last addition, the argon barbotage was stopped and the reaction allowed to continue for 50 minutes under gentle agitation.

After cooling to ambient temperature, the resulting product was purified by dialysis, and then lyophilized in order to obtain the polymer of the invention.

### Example 6

### Synthesis of a Block Polymer: Chitosane 70-Methyl Polyacrylate-Methyl Polymethacrylate

2.2 g of chitosane 70 were dissolved in 32 ml of nitric acid at 0.02M in a 250-ml triple neck flask equipped with a condenser and an agitator, with argon barbotage, the flask being plunged into a 60° C. water bath.

After 10 minutes, 25 ml of a 0.08M solution of ceric ammonium nitrate (IV) in 0.2M of nitric acid and 7 ml of methyl acrylate were added under high agitation.

After 20 minutes, 25 ml of a 0.08M solution of ceric ammonium nitrate (IV) in 0.2M of nitric acid and 7 ml of methyl methacrylate were added under high agitation.

Twenty minutes after the last addition, the argon barbotage was stopped and the reaction allowed to continue for 50 minutes under gentle agitation.

After cooling to ambient temperature, the resulting product was purified by dialysis, and then lyophilized in order to obtain the polymer of the invention. Study of Release of an Active Principal In Vitro

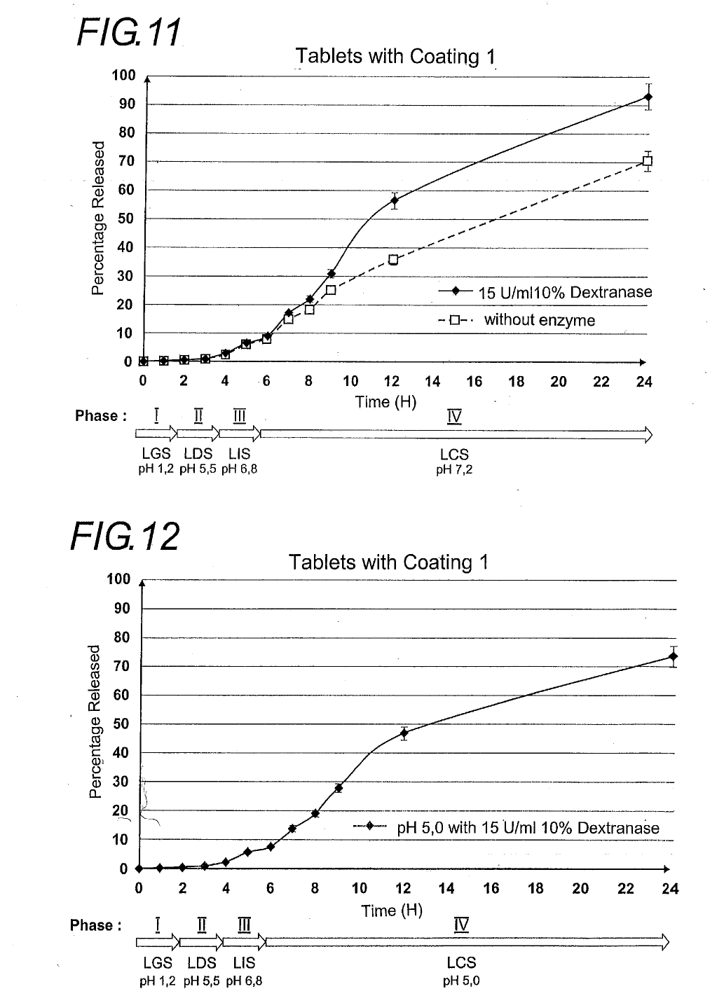

In order to demonstrate the effectiveness of a polymer according to the invention, a series of tests was conducted by the inventors which will now be described with reference to FIGS. 11 through 16.

To perform these tests, the inventors prepared tablets containing theophylline as the active principal and coated them with different block copolymers according to the invention. They then studied the in vitro release of the active principal contained in the galenic forms thus obtained throughout the digestive tract of a simulated digestive system.

The tablets tested were obtained conventionally by wet granulation from theophylline and a group of excipients according to the detailed composition in the table below:

| | | Percentage by Mass |
|---|---|---|
| | Quantity for a Tablet | Relative to |
| Composition | (mg) | Tablet Weight |
| Anhydrous Theophylline | 100 | 26.66 |
| Lactose | 183.25 | 48.87 |
| Avicel ® PH101 | 73 | 19.47 |
| Wheat Amidine | 15 | 4 |
| Magnesium Stearate | 3.75 | 1 |
| Total Weight | 375 | 100 |
| Compressibility Index | 12.19% | (≦20%) |

The mixture was compressed in order to obtain tablets having the following characteristics: uniform mass, hardness, disaggregating behavior, friability, and theophylline content. The measurements were performed using the Pharmacopée Européene, 6th Ed., 2007 as a reference. The results obtained are shown in the table below. These are average values.

| | | | | Active |
|---|---|---|---|---|
| | | | | Principal |
| Uniformity of | | Disaggregation | Friability | Content |
| Mass (mg) | Hardness (N) | (min) | (% Mass) | (% Mass) |
| 380.05 | 115.6 | <1 | 0.23 | 100 |

The tablets obtained were then covered with a coating containing a block copolymer according to the invention. In order to demonstrate the effectiveness of all the copolymers according to the invention, three coating dispersions were prepared from three different copolymers. The first dispersion, called coating 1, contains a dextran 70 copolymer-methyl polyacrylate-methyl polymethacrylate-polymethacrylic acid; the second dispersion, called coating 2, contains a dextran 70 copolymer-methyl polyacrylate-polymethacrylic acid; and the third dispersion, called coating 3, contains a dextran 10 copolymer-methyl polyacrylate-methyl polymethacrylate-polymethacrylic acid. The composition of these three coating dispersions is detailed in the table below:

| | Coating 1 | Coating 2 | Coating 3 |
|---|---|---|---|
| | (Dex70-MA-MMA- | (Dex70-MA- | (Dex10-MA- |
| Composition (g) | MAA) | MAA) | MMA-MAA) |
| Block Copolymer | 50 | 50 | 50 |
| (dry state) | | | |
| Talc | 15 | 15 | 15 |
| Triethyl Citrate | 10 | 10 | 10 |
| Silicon Emulsion | 1 | 1 | 1 |
| Water | 7.6 | 7.6 | 7.6 |
| Dry Residue | 7.6 | 7.6 | 7. |
| Content | | | |
| (% Mass) | | | |

The tablets were coated with one of these three dispersion coatings by pulverization using a fluidized bed. They were then dried using hot air and then a laboratory oven. At the end of the coating process, the inventors obtained three groups of tablets distinguished by their coatings. Relative to the original uncoated tablets, the coated tablets showed an average weight gain respectively equal to 10% for coating 1, the tablets with a dextran 70-methyl polyacrylate-methyl polymethacrylate-polymethacrylic acid based coating; 8.6% for coating 2, the tablets covered with a dextran 70-methyl polyacrylate-polymethacrylic acid based coating; and 11% for coating 3, the tablets covered with a dextran 10-methyl polyacrylate-methyl polymethacrylate-polymethacrylic acid based coating.

These three groups of coated tablets were used to study the release of theophylline during its trajectory through the digestive system. This study took place in vitro with the help of a dissolution tester of the type USP III (BIO-DIS™, RRT9, Caleva, Ltd., United Kingdom) for reproducing the changes in pH encountered along the digestive tract.

The study unfolded in four consecutive phases called phases I through IV, during which the experimental surround successively simulated gastric liquid (simulated gastric liquid LGS), duodenal liquid (simulated duodenal liquid LDS), intestinal liquid (simulated intestinal liquid LIS), and colonic liquid (simulated colonic liquid LCS). For colonic liquid (LCS), several variations were tested, namely with a pH of 7.2, a pH of 7.2 in the presence of dextranase (15 U/ml), and with a pH of 5.0 in the presence of dextranase (15 U/ml). All the parameters of this study are grouped in the table below.

| | Phase | | | |
|---|---|---|---|---|
| | Phase I | Phase II | Phase III | Phase IV |
| | LGS | LDS | LIS | LCS |
| Volume of experimental | 250 | 250 | 250 | 250 |
| surround (ml) | | | | |
| pH | 1.2 | 5.5 | 6.8 | 7.2 or 5.0 |
| Duration (hours) | 2 | 2 | 2 | 18 |
| Amplitude | 20 | 20 | 20 | 5 |
| (Number of vertical | | | | |
| movements from bottom to | | | | |
| top and from top to | | | | |
| bottom/min.) | | | | |

Samples of 5 ml were drawn at regular intervals without replacing the dissolution surround. These samples were diluted appropriately and analyzed using a 272 nm UV-Visible spectrophotometer to determine the quantity of theophylline released during each phase. This study of release was repeated three times.

For each group of tablets the average quantity of theophylline released over time is shown by the graphs in FIGS. 11 through 16. It is expressed as a percentage of theophylline released relative to total theophylline contained in the tablets at the outset.

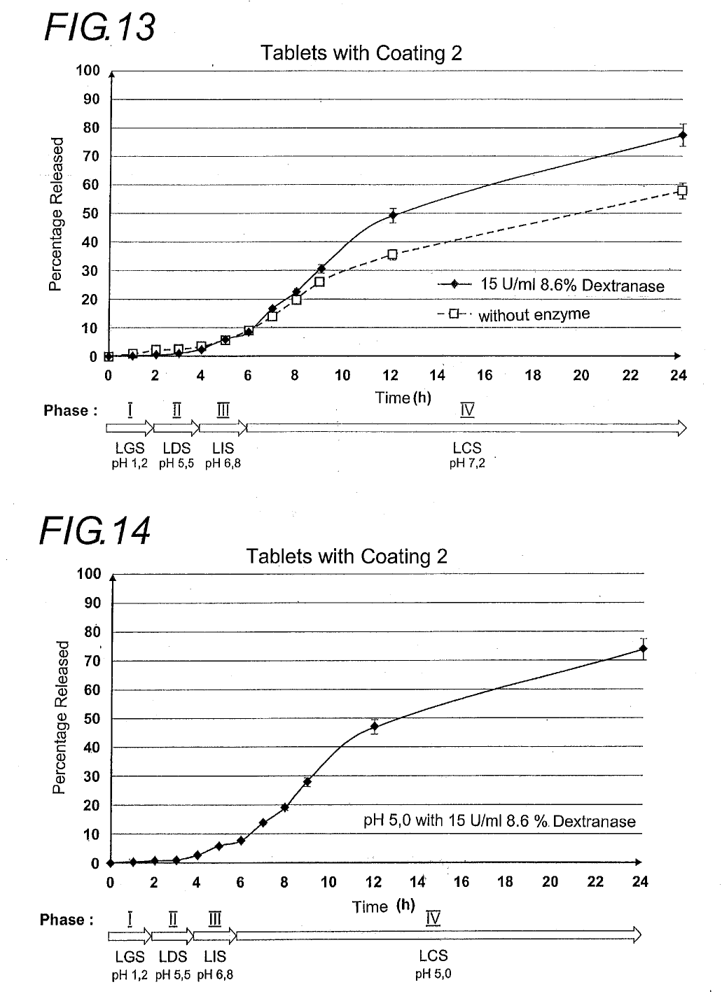

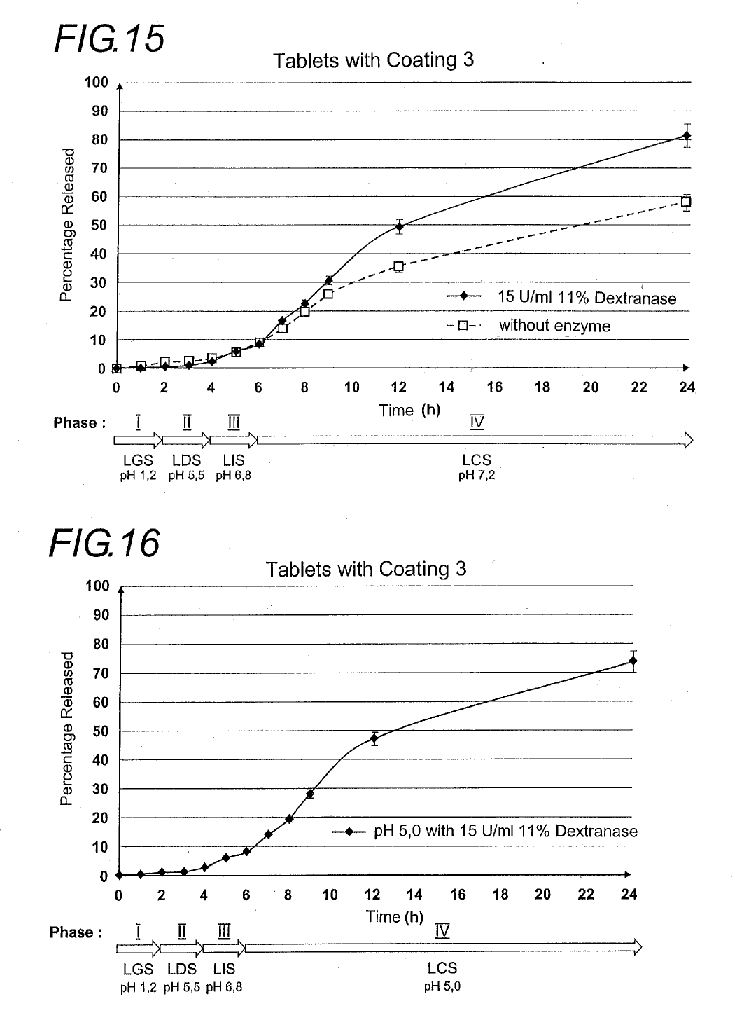

FIGS. 11 and 12 show the average quantity of theophylline released over time by the tablets with coating 1; FIGS. 13 and 14, by the tablets with coating 2; and FIGS. 15 and 16, by the tablets with coating

From studying these graphs, it is apparent that regardless of which inventive coating is used, hardly any theophylline is released at the level of the stomach (Phase I) and the duodenum (Phase II) and there is only a very slight release of theophylline, less than 10%, in the first part of the intestine (Phase III). The polymer of the invention therefore maintains the active principal intact until it reaches the colon.

When the tablets reach the level of the colon (Phase IV), a rapid increase in the release of theophylline is noted, which continues throughout the tablets' trajectory through the colon and ends with very large amounts of theophylline being released after the passage of 24 hours, more than 70% in the presence of dextranase, and even on the order of 80% for coating 2 or 3, or more than 90% for coating 1 in the presence of dextranase and with the colon at the normal pH of 7.2.

Release of the active principal occurs when the pH of the surround is essentially neutral, which corresponds to normal colon pH, by dissolution of the polyacrylic blocks even in the absence of enzymes. However, this release is greatly improved in the presence of enzymes such a dextranase produced by colonic microflora, which also dissolve the polypeptide block of the polymer according to the invention. The polymer of the invention is therefore more effective than a polymer having only an acrylic structure when the pH condition of the colon is normal.

FIGS. 12, 14 and 16 it should be noted that a considerable release of theophylline also occurs in phase IV when the pH of the surround is equal to 5 in the presence of dextranase by dissolution of the polypeptide block of the polymer according to the invention. In such an acetic milieu, often encountered in colon disease, a polymer with an acrylic structure alone would be completely ineffective.

As these experiments demonstrate, the polymer-based coatings of the invention that were tested fulfill their functions completely: they protect the active principal until it reaches the colon and cause this active principal to be almost completely released once it reaches the colon, whether the colon pH is generally neutral or even if the pH is acetic.

It is obvious that the invention is not limited to the preferred embodiments described above and shown in the various drawings, since a person skilled in the art could conceive of numerous modifications and variations without departing from the realm or the scope of the invention defined in the claims.

1-15. (canceled)

16. An oral galenic form (1) containing at least one active principal (9) to be released specifically within the colon (7) and containing a polymer for colon-specific release and designed to protect the at least one active principal (9) until the at least one active principal (9) reaches the colon (7) and release the at least one active principal (9) specifically within the colon (7),
wherein the polymer for colon-specific release is a block copolymer comprising a polysaccharide block (17) that is degraded by colonic microflora irrespective of a pH of the colon, and
at least two hydrophobic polyacrylic blocks (18) allow the at least one active principal (9) to remain intact until the at least one active principal (9) reaches the colon (7), and the polyacrylic blocks (18) are grafted one after the other onto the polysaccharide block (17) and are soluble at a generally neutral pH.

17. The oral galenic form (1) according to claim 16, wherein the polysaccharide block (17) is made from one of dextran, guar gum, amylase, amidine, chitosane and pectin.

18. The oral galenic form (1) according to claim 16, wherein the polymer for colon-specific release comprises either two or three polyacrylic blocks (18).

19. The oral galenic form (1) according to claim 1, wherein polyacrylic block (18) is a chain of at least one of methyl polyacrylate (19), methyl polymethacrylate (20) and polymethacrylic acid (21).

20. The oral galenic form (1) according to claim 18, wherein the polymer for colon-specific release comprises, following the polysaccharide block, (17):
a methyl polyacrylate block (19), a methyl polymethacrylate block (20) and a polymethacrylic acid block (21); or
a methyl polyacrylate block (19) and a methyl polymethacrylate block (20); or
a methyl polyacrylate block (19) and a polymethacrylic acid block (21); or
a methyl polymethacrylate block (20) and a polymethacrylic acid block (21).

21. The oral galenic form (1) according to claim 16, wherein the oral galenic form (1) is one of a tablet (11), a gel capsule (15), a granule (14) and a microgranule.

22. A method of producing a polymer for colon-specific release of an oral galenic form (1) containing at least one active principal (9) to be released specifically within the colon (7) and containing a polymer for colon-specific release and designed to protect the at least one active principal (9) until the at least one active principal (9) reaches the colon (7) and release the at least one active principal (9) specifically within the colon (7), wherein the polymer for colon-specific release is a block copolymer comprising a polysaccharide block (17) that is degraded by colonic microflora irrespective of a pH of the colon, and at least two hydrophobic polyacrylic blocks (18) allow the at least one active principal (9) to remain intact until the at least one active principal (9) reaches the colon (7), and the polyacrylic blocks (18) are grafted one after the other onto the polysaccharide block (17) and are soluble at a generally neutral pH, the method comprising the step of:
obtaining the polyacrylic blocks (18) by successively adding different acrylic monomers to the polysaccharide block (17) during several successive steps of radical polymerization in emulsion.

23. The method of producing according to claim 22, further comprising the step of selecting the acrylic monomers from the following group consisting of and in the following relative order: methyl acrylate, methyl methacrylate and methacrylic acid.

24. The method of producing according to claim 22, further comprising the step of performing the successive steps of radical polymerization in an emulsion in a presence of cerium (IV), argon and nitric acid and at a temperature of about 60° C.

25. The method of producing according to claim 24, further comprising the step of using a range of concentration of cerium (IV) from between 0.001 to 0.02 mol/l.

26. The use of a polymer released specifically within the colon to produce an oral galenic form (1) containing at least one active principal (9) which must be released specifically within the colon (7), the polymer for colon-specific release being designed to protect the at least one active principal (9) until the at least one active principal (9) reaches the colon (7) and to release the at least one active principal (9) specifically within the colon (7),
wherein the polymer for colon-specific release is a block copolymer comprising a polysaccharide block (17) that is degraded by the colonic microflora regardless of a pH of a colon, and
at least two hydrophobic polyacrylic blocks (18) which allow the polymer to remain intact until the at least one active principal (9) reaches the colon (7),
the polyacrylic blocks (18) are grafted one after the other onto the polysaccharide block (17) and are soluble at a generally neutral pH.

27. The use according to claim 26, wherein the polymer for colon-specific release is used to produce a coating (13).

28. The use according to claim 27, wherein the thickness of the coating (13) ranges from about 1 μm to about 1 mm.

29. The use according to claim 27, wherein the coating (13), in addition to the polymer for colon-specific release, contains at least one of a plastifying agent, an element having an insoluble structure regardless of pH and a supplemental substance.

30. The use according to claim 26, wherein the polymer for colon-specific release is used as a matrix agent.
